(19) United States
(12) Patent Application Publication
(10) Pub. No.: US 2014/0287414 A1
(43) Pub. Date: Sep. 25, 2014

(54) SYSTEM AND METHOD FOR ANALYZING DNA USING APPLICATION OF MOBILE DEVICE

(71) Applicant: Electronics and Telecommunications Research Institute, Daejeon (KR)

(72) Inventors: Kwang Hyo CHUNG, Daejeon (KR); Jin Tae KIM, Daejeon (KR); Doo Hyeb YOUN, Daejeon (KR); Ki-Chul KIM, Daejeon (KR); Young-Jun YU, Daejeon (KR); Jin Sik CHOI, Daejeon (KR); Choon Gi CHOI, Daejeon (KR)

(73) Assignee: Electronics and Telecommunications Research Institute, Daejeon (KR)

(21) Appl. No.: 14/012,276

(22) Filed: Aug. 28, 2013

(30) Foreign Application Priority Data
Mar. 20, 2013 (KR) 10-2013-0030014

(51) Int. Cl. C12Q 1/68

(52) U.S. Cl. CPC C12Q 1/686
USPC 435/6.12; 435/287.2

(57) ABSTRACT

A DNA analysis system that controls DNA analysis by wireless using an application of a mobile device and a very small DNA analysis apparatus, and that receives a DNA analysis result in real time on the spot is provided. Therefore, by performing DNA analysis by simultaneously controlling a plurality of small DNA analysis apparatuses using signal processing and screen display functions of a mobile device, analysis speed of DNA is improved, and an analysis result of DNA can be provided in real time. Further, by forming a DNA analysis apparatus in a very small size, DNA can be immediately analyzed with low power consumption on the spot using a small sample, and the DNA analysis apparatus can be carried.

### CROSS-REFERENCE TO RELATED APPLICATION

This application claims priority to and the benefit of Korean Patent Application No. 1 0-201 3-003001 4 filed in the Korean Intellectual Property Office on Mar. 20, 2013, the entire contents of which are incorporated herein by reference.

### BACKGROUND OF THE INVENTION

(a) Field of the Invention

The present invention relates to a method and apparatus for analyzing DNA using an application of a mobile device.

(b) Description of the Related Art

In order to analyze deoxyribonucleic acid (DNA) within a sample, a process of amplifying DNA and a process of analyzing DNA are necessary. Polymerase chain reaction (PCR) is typical of methods for amplifying DNA, and in a process of analyzing DNA, an electrical method, an electrochemical method, and an optical method are representatively used.

In this case, PCR is a method of amplifying DNA by repeatedly performing constant temperature cycling in a sample and is performed with denaturation that separates a DNA double helix, annealing in which DNA template adjusts to search for a complementary pair, and extension in which DNA grows, and each thereof is performed at different temperatures.

DNA analysis through such amplification and analysis of DNA is generally provided to one apparatus or a plurality of apparatuses that can perform a series of processes, and real-time PCR apparatus is a typical example.

The apparatuses are generally provided as a laboratory desktop type, and currently, a DNA analysis apparatuses are formed with a small size. When a DNA analysis apparatus is formed with a small size, there are merits that DNA can be analyzed on the spot, only a small sample needs to be used, the DNA analysis apparatus can be conveniently used through portability, and it can be used with low power consumption.

However, because precise temperature control is necessary in a DNA amplification process such as PCR, a heater, a heating block, a temperature sensor, and a module for temperature control are necessary. Further, for DNA analysis, because an analysis sensor, a module, and a user interface for signal processing are necessary, it is difficult to form an apparatus that forms individual modules in one integral body in a small size due to a limitation according to an individual module size and a limitation according to mutual interfacing between individual modules.

### SUMMARY OF THE INVENTION

The present invention has been made in an effort to provide a DNA analysis system having advantages of controlling DNA analysis by wireless using an application of a mobile device and a very small DNA analysis apparatus and receiving a DNA analysis result in real time on the spot.

An exemplary embodiment of the present invention provides a DNA analysis module that is controlled through an application of a mobile device. The DNA analysis module includes: a plastic chip including a substrate that injects an analysis sample including DNA, a plurality of heaters that heat the analysis sample, a temperature sensor that measures temperature of the analysis sample, a DNA sensor that analyzes DNA that is included in the analysis sample, and a chip electrode that is electrically connected to the plurality of heaters, the temperature sensor, and the DNA sensor; and a transmitting/receiving kit that is electrically connected to the plastic chip to transmit/receive a signal and that transmits and receives a signal to and from the mobile device through wireless communication.

The temperature sensor may transfer temperature information of the analysis sample to the transmitting/receiving kit, the DNA sensor may transfer an analysis result of the DNA to the transmitting/receiving kit, and the transmitting/receiving kit may transmit the temperature information and the analysis result of the DNA to the mobile device.

The transmitting/receiving kit may transfer a control signal of the plurality of heaters received from the mobile device to the plastic chip, and the plurality of heaters may heat the analysis sample according to the control signal. The substrate may be produced through an injection molding, hot embossing, or ultraviolet ray shaping process.

The plurality of heaters, the temperature sensor, the DNA sensor, and the chip electrode may be produced by a method of patterning gold, platinum, mercury, aluminum, chrome, graphene, or carbon nanotubes at a surface of an electrode substrate.

The DNA sensor may analyze DNA by an electrical resistance detection method, an electrochemical detection method, or a fluorescence detection method.

The plurality of heaters, the temperature sensor, the DNA sensor, and the chip electrode may be bonded to the substrate by a hot bonding, ultraviolet (UV) bonding, adhesive bonding, or plasma bonding method.

The plastic chip and the transmitting/receiving kit may be electrically connected through a slot insertion type connection, pin connection, wire bonding, soldering, or silver paste bonding method.

The transmitting/receiving kit may include: a kit electrode that electrically connects the transmitting/receiving kit to the plastic chip; a signal processor that processes a signal received from the plastic chip and a signal received from the mobile device through wireless communication through at least one of an analog to digital converter (ADC), a digital to analog converter (DAC), a filter, and a field programmable gate array (FPGA); and a signal transmitting/receiving unit that transmits and receives a signal to and from the mobile device through wireless communication.

Another embodiment of the present invention provides a DNA analysis system that can analyze DNA through wireless communication. The DNA analysis system includes: a plurality of DNA analysis modules including a substrate that injects an analysis sample including DNA, a plurality of heaters that heat the analysis sample, a temperature sensor that measures temperature of the analysis sample, and a DNA sensor that analyzes DNA that is included in the analysis sample; and a mobile device in which an application that transmits and receives a signal to and from the plurality of DNA analysis modules through wireless communication to control the plurality of DNA analysis modules is installed.

The mobile device may control at least one of the plurality of DNA analysis modules and compares DNA analysis results received from the at least one DNA analysis module.

Yet another embodiment of the present invention provides a method of analyzing DNA of a DNA analysis module operating according to a control signal of a mobile device. The method includes: receiving a control signal of the DNA analysis module from the mobile device and heating the analysis sample; analyzing amplified DNA in the heated analysis sample; and transmitting an analysis result of the DNA to the mobile device.

### BRIEF DESCRIPTION OF THE DRAWINGS

FIG. 1 is a diagram illustrating a DNA analysis system according to an exemplary embodiment of the present invention.

FIG. 2 is a diagram illustrating a plastic chip according to an exemplary embodiment of the present invention.

FIG. 3 is a diagram illustrating a transmitting/receiving kit according to an exemplary embodiment of the present invention.

FIG. 4 is a diagram illustrating an application of a mobile device according to an exemplary embodiment of the present invention.

FIG. 5 is a diagram illustrating a network that is formed between a plurality of plastic chips and a transmitting/receiving kit, and a mobile device according to an exemplary embodiment of the present invention.

FIG. 6 is a flowchart illustrating a DNA analysis method according to an exemplary embodiment of the present invention.

### DETAILED DESCRIPTION OF THE EMBODIMENTS

In the following detailed description, only certain exemplary embodiments of the present invention have been shown and described, simply by way of illustration. As those skilled in the art would realize, the described embodiments may be modified in various different ways, all without departing from the spirit or scope of the present invention. Accordingly, the drawings and description are to be regarded as illustrative in nature and not restrictive. Like reference numerals designate like elements throughout the specification.

In addition, in the entire specification, unless explicitly described to the contrary, the word “comprise” and variations such as “comprises” or “comprising” will be understood to imply the inclusion of stated elements but not the exclusion of any other elements. In addition, the terms “-er”, “-or”, “module”, and “block” described in the specification mean units for processing at least one function and operation and can be implemented by hardware components or software components and combinations thereof.

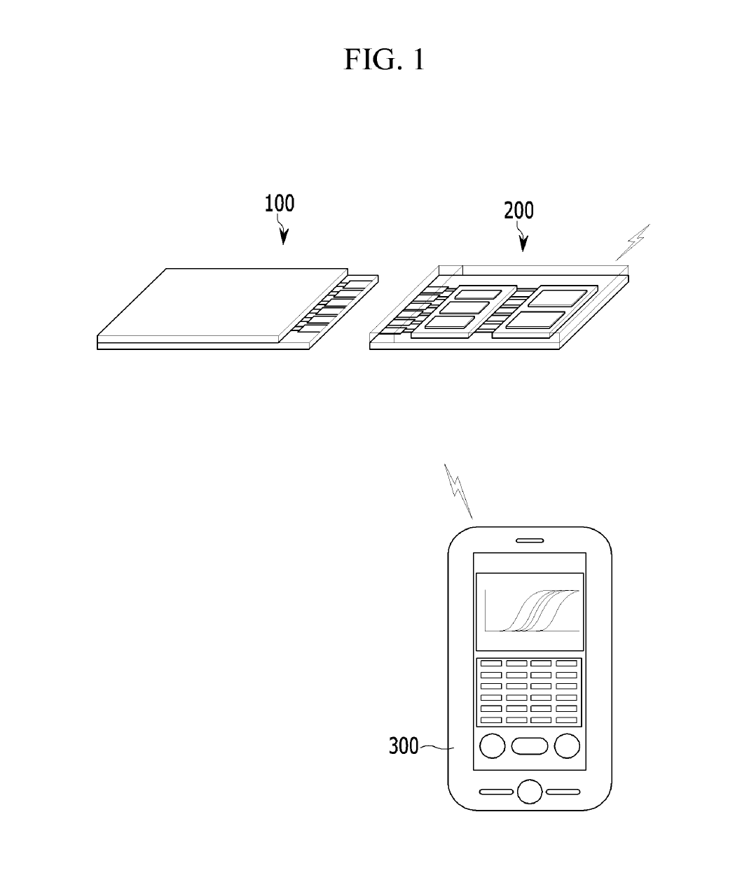

FIG. 1 is a diagram illustrating a DNA analysis system according to an exemplary embodiment of the present invention.

Referring to FIG. 1, the DNA analysis system according to an exemplary embodiment of the present invention includes a plastic chip 100, a transmitting/receiving kit 200, and a mobile device 300 in which an application is installed.

The plastic chip 100 amplifies DNA and analyzes the amplified DNA.

The transmitting/receiving kit 200 transmits an electrical signal received from the plastic chip 100 to the mobile device 300, and transmits the electrical signal received from the mobile device 300 to the plastic chip 100.

The mobile device 300 controls amplification and analysis of DNA in real time through an application. In this case, the mobile device 300 includes wireless communication equipment of a smart phone, a tablet PC, and a personal digital assistant (PDA).

By connecting the plastic chip 100 and the transmitting/receiving kit 200, a user of the DNA analysis system according to an exemplary embodiment of the present invention can carry them in one module.

The transmitting/receiving kit 200 and the mobile device 300 transmit/receive a wireless signal using near field communication (NFC), ZigBee, Bluetooth, ultra-wideband (UWB), or WiFi technology.

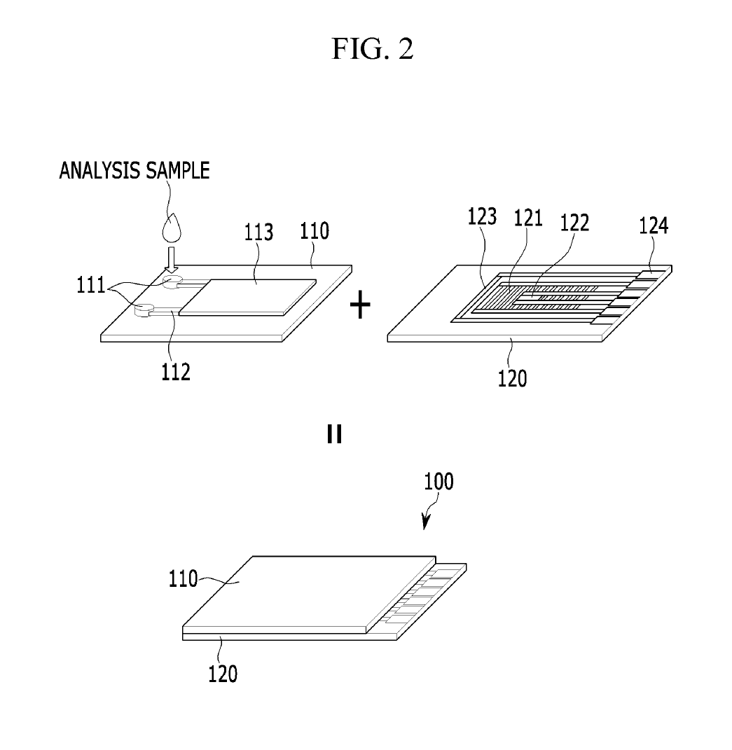

FIG. 2 is a diagram illustrating a plastic chip according to an exemplary embodiment of the present invention.

Referring to FIG. 2, the plastic chip 100 according to an exemplary embodiment of the present invention includes a flow channel substrate 110 and an electrode substrate 120. The flow channel substrate 110 includes a plurality of sample inlets 111, a microchannel 112, and a microchamber 113. The electrode substrate 120 includes a plurality of microheaters 121, a temperature sensor 122, a DNA sensor 123, and a chip electrode 124.

When an analysis sample contacts the sample inlet 111, the analysis sample is injected into the microchamber 113 through the microchannel 112. In this case, a quantity of the analysis sample is generally 1 ul-10 ul, and may be changed according to a kind of DNA to be used as an analysis target.

The flow channel substrate 110 may be produced in a plastic chip production process such as injection molding, hot embossing, and ultraviolet ray shaping.

The electrode substrate 120 heats an analysis sample that is collected at the microchamber 113 through the plurality of microheaters 121 and measures a temperature through the temperature sensor 122. Further, the electrode substrate 120 analyzes amplified DNA through the DNA sensor 123. In this case, the DNA sensor 123 measures amplified DNA with an electrical resistance detection method, an electrochemical detection method, or a fluorescence detection method.

The microheater 121, the temperature sensor 122, the DNA sensor 123, and the chip electrode 124 of the electrode substrate 120 may be produced with a method of patterning a metal such as gold (Au), platinum (Pt), mercury (Ag), aluminum (Al), and chrome (Cr), or a carbon material such as graphene and carbon nanotubes (CNT), at a surface of the electrode substrate 120.

The electrode substrate 120 heats an analysis sample of the microchamber 113 using the plurality of microheaters 121, the temperature sensor 122, the DNA sensor 123, and the chip electrode 124, and measures a signal.

The flow channel substrate 110 and the electrode substrate 120 are bonded through a plastic chip bonding process of hot bonding, UV bonding, adhesive bonding, and plasma bonding.

The plastic chip 100 may be formed with polymethyl methacrylate (PMMA), polycarbonate (PC), cyclo olefin copolymer (COC), polyamide (PA), polyethylene (PE), polypropylene (PP), polyphenylene ether (PPE), polystyrene (PS), polyoxymethylene (POM), polyetheretherketone (PEEK), polytetrafluoroethylene (PTFE), polyvinylchloride (PVC), polyvinylidene fluoride

(PVDF), polybutylene terephthalate (PBT), fluorinated ethylenepropylene (FEP), and perfluoralkoxyalkane (PFA). Further, by biologically, chemically, or optically processing a surface of the plastic chip 100, a component such as an enzyme, a protein, and DNA that are included in a sample may be prevented from being adsorbed to a surface.

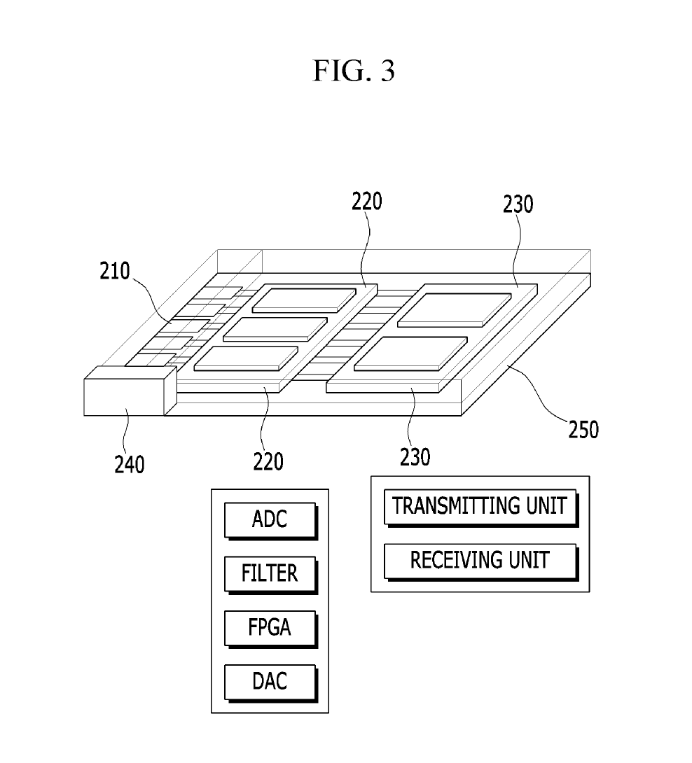

FIG. 3 is a diagram illustrating a transmitting/receiving kit according to an exemplary embodiment of the present invention.

Referring to FIG. 3, the transmitting/receiving kit 200 according to an exemplary embodiment of the present invention includes a kit electrode 210, a signal processor 220, a signal transmitting/receiving unit 230, a power supply unit 240, and a printed circuit board (PCB) 250.

The kit electrode 210 electrically connects the plastic chip 100 and the transmitting/receiving kit 200 with a method of slot insertion connection, pin connection, wire bonding, soldering, and silver paste bonding.

The signal processor 220 processes a signal that is transmitted from the DNA sensor 123 of the plastic chip 100, transfers the signal to the signal transmitting/receiving unit 230, processes the signal received from the mobile device 300, and transfers the signal to the plastic chip 100. In this case, the transmitted/received signal is processed through an analog to digital converter (ADC), a filter, a field programmable gate array (FPGA), and a digital to analog converter (DAC).

The signal transmitting/receiving unit 230 transmits/receives a wireless signal between the transmitting/receiving kit 200 and the mobile device 300 using near field communication (NFC), ZigBee, Bluetooth, ultra-wideband (UWB), or WiFi technology.

The power supply unit 240 supplies power to the plastic chip 100 and the transmitting/receiving kit 200. The user should be able to carry one DNA analysis module including the plastic chip 100 and the transmitting/receiving kit 200, and because the DNA analysis module consumes less power, the power supply unit 240 can be formed in a small size. That is, the power supply unit can be formed as a disposable battery or a rechargeable battery.

The kit electrode 210, the signal processor 220, the signal transmitting/receiving unit 230, and the power supply unit 240 of the transmitting/receiving kit 200 may be installed on the PCB 250, and the PCB 250 should have a sufficiently wide size to connect to the plastic chip 100.

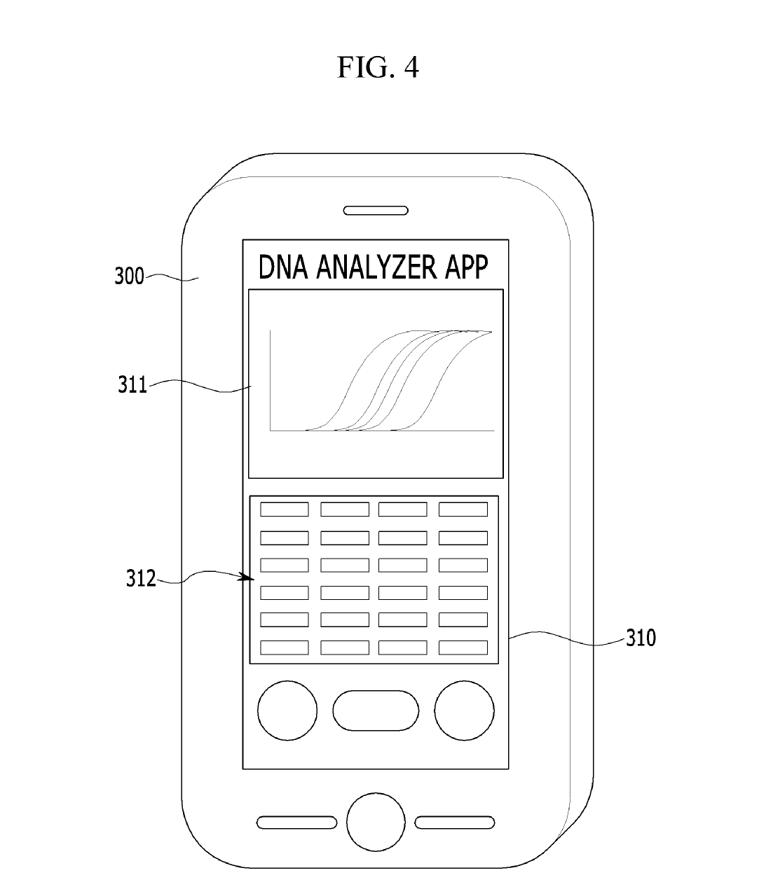

FIG. 4 is a diagram illustrating a DNA analysis application of a mobile device according to an exemplary embodiment of the present invention.

Referring to FIG. 4, the DNA analysis application 310 that is installed in the mobile device 300 according to an exemplary embodiment of the present invention includes a signal processing control and display unit 311 and a user interface 312.

The signal processing control and display unit 311 transmits and receives a signal to and from the transmitting/receiving kit 200, and amplifies the signal, removes noise of the signal, and stores and controls the signal received from the transmitting/receiving kit 200, and displays a result on a screen of the mobile device 300.

The user interface 312 transfers an input of a user, having determined a DNA amplification and analysis process that is displayed on the screen, to the signal processing control and display unit 311.

That is, an application according to an exemplary embodiment of the present invention amplifies the signal received from the transmitting/receiving kit 200, displays a DNA amplification and analysis process on the screen of the mobile device 300, and enables the user to control the DNA amplification and analysis process through a user interface.

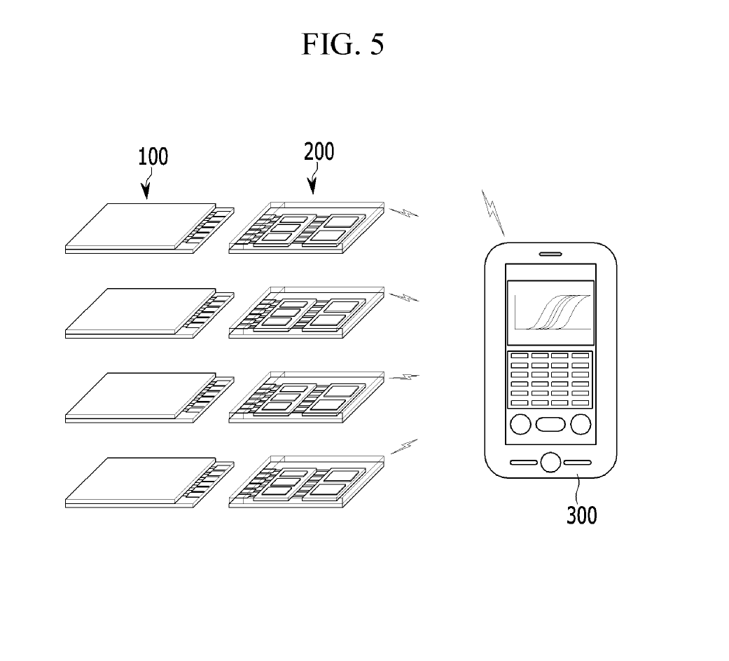

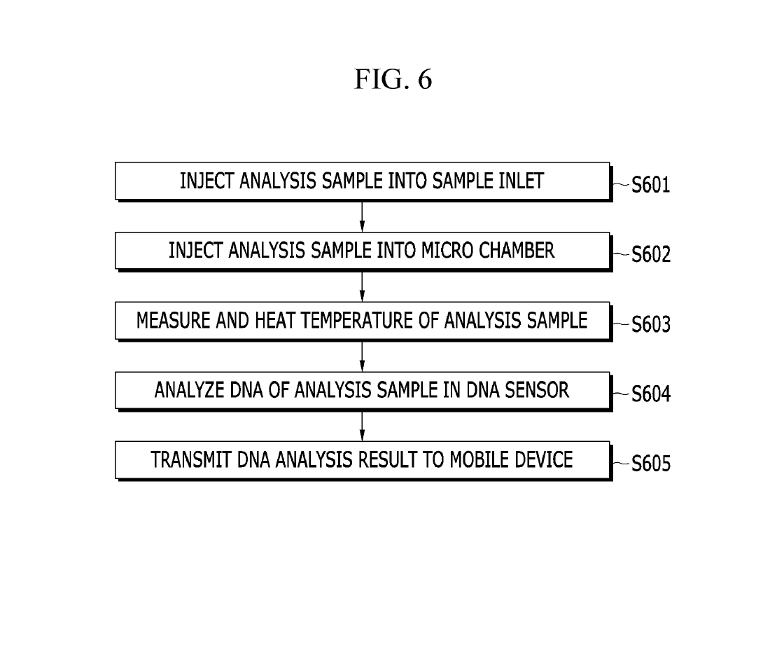

FIG. 5 is a diagram illustrating a network that is formed between a plurality of plastic chips, a transmitting/receiving kit, and a mobile device according to an exemplary embodiment of the present invention, and FIG. 6 is a flowchart illustrating a DNA analysis method according to an exemplary embodiment of the present invention.

Referring to FIGS. 5 and 6, a user using a DNA analysis system according to an exemplary embodiment of the present invention injects an analysis sample in which DNA analysis is requested into the sample inlet 111 (S601).

The analysis sample that is injected into the sample inlet 111 is injected into the microchamber 113 through the microchannel 112 (S602).

Thereafter, the temperature of the analysis sample that is collected at the microchamber 113 is measured by the temperature sensor 122, and the analysis sample is heated by the microheater 121 (S603).

The user controls the temperature of an analysis sample according to a temperature profile that is requested at each step (denaturation, annealing, and extension) of PCR using an application of the mobile device 300.

In this case, the chip electrode 124 that is electrically connected to the kit electrode 210 of the transmitting/receiving kit 200 transfers temperature information of the analysis sample to the transmitting/receiving kit 200 and transfers a microheater control signal received from the mobile device 300 to the microheater 121. Further, the signal processor 220 of the transmitting/receiving kit 200 processes a signal received from the kit electrode 210, transfers the signal to the signal transmitting/receiving unit 230, processes the signal received from the signal transmitting/receiving unit 230, and transfers the signal to the plastic chip 100.

Therefore, the user determines temperature information of the analysis sample through an application of the mobile device, controls the microheater 121 based on the temperature information of the analysis sample, and performs PCR of the analysis sample.

Thereafter, DNA of the analysis sample that is amplified by PCR is analyzed in the DNA sensor 123 (S604). In this case, the DNA sensor 123 analyzes amplified DNA by an electrical resistance detection method, an electrochemical detection method, or a fluorescence detection method.

Thereafter, a DNA analysis result is transferred to the transmitting/receiving kit 200 through the chip electrode 124, signal processing thereof is performed, and the DNA analysis result is transmitted to the mobile device 300 (S605). Finally, the user can know the DNA analysis result through an application of the mobile device 300.

Alternatively, the user controls a plurality of plastic chips 100 and the transmitting/receiving kit 200 using an application of the mobile device 300, thereby simultaneously analyzing DNA and comparing results thereof.

In this way, according to an exemplary embodiment of the present invention, by performing DNA analysis by simultaneously controlling a plurality of small-size DNA analysis apparatuses actively using signal processing and a screen display function of a mobile device, analysis speed of DNA can be improved and an analysis result of DNA can be provided in real time. Further, by forming a DNA analysis apparatus in a very small size, DNA can be immediately analyzed with low power consumption on the spot using a small sample, and the DNA analysis apparatus can be carried.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

What is claimed is:

1. A DNA analysis module that is controlled through an application of a mobile device, the DNA analysis module comprising:
a plastic chip comprising a substrate that injects an analysis sample comprising DNA, a plurality of heaters that heat the analysis sample, a temperature sensor that measures temperature of the analysis sample, a DNA sensor that analyzes DNA that is included in the analysis sample, and a chip electrode that is electrically connected to the plurality of heaters, the temperature sensor, and the DNA sensor; and
a transmitting/receiving kit that is electrically connected to the plastic chip to transmit/receive a signal and that transmits and receives a signal to and from the mobile device through wireless communication.

2. The DNA analysis module of claim 1, wherein the temperature sensor transfers temperature information of the analysis sample to the transmitting/receiving kit, the DNA sensor transfers an analysis result of the DNA to the transmitting/receiving kit, and
the transmitting/receiving kit transmits the temperature information and the analysis result of the DNA to the mobile device.

3. The DNA analysis module of claim 1, wherein the transmitting/receiving kit transfers a control signal of the plurality of heaters received from the mobile device to the plastic chip, and
the plurality of heaters heat the analysis sample according to the control signal.

4. The DNA analysis module of claim 1, wherein the substrate is produced through an injection molding, hot embossing, or ultraviolet ray shaping process.

5. The DNA analysis module of claim 1, wherein the plurality of heaters, the temperature sensor, the DNA sensor, and the chip electrode are produced by a method of patterning gold, platinum, mercury, aluminum, chrome, graphene, or carbon nanotubes at a surface of an electrode substrate.

6. The DNA analysis module of claim 1, wherein the DNA sensor analyzes DNA by an electrical resistance detection method, an electrochemical detection method, or a fluorescence detection method.

7. The DNA analysis module of claim 1, wherein the plurality of heaters, the temperature sensor, the DNA sensor, and the chip electrode are bonded to the substrate by a hot bonding, ultraviolet (UV) bonding, adhesive bonding, or plasma bonding method.

8. The DNA analysis module of claim 1, wherein the plastic chip and the transmitting/receiving kit are electrically connected through a slot insertion connection, pin connection, wire bonding, soldering, or silver paste bonding method.

9. The DNA analysis module of claim 1, wherein the transmitting/receiving kit comprises:
a kit electrode that electrically connects the transmitting/receiving kit to the plastic chip;
a signal processor that processes a signal received from the plastic chip and a signal received from the mobile device through wireless communication through at least one of an analog to digital converter (ADC), a digital to analog converter (DAC), a filter, and a field programmable gate array (FPGA); and
a signal transmitting/receiving unit that transmits and receives a signal to and from the mobile device through wireless communication.

10. A DNA analysis system that can analyze DNA through wireless communication, the DNA analysis system comprising:
a plurality of DNA analysis modules comprising a substrate that injects an analysis sample comprising DNA, a plurality of heaters that heat the analysis sample, a temperature sensor that measures temperature of the analysis sample, and a DNA sensor that analyzes DNA that is included in the analysis sample; and
a mobile device in which an application that transmits and receives a signal to and from the plurality of DNA analysis modules through wireless communication to control the plurality of DNA analysis modules is installed.

11. The DNA analysis system of claim 10, wherein the mobile device controls at least one of the plurality of DNA analysis modules and compares DNA analysis results received from the at least one DNA analysis module.

12. A method of analyzing DNA of a DNA analysis module operating according to a control signal of a mobile device, the method comprising:
receiving a control signal of the DNA analysis module from the mobile device and heating the analysis sample;
analyzing amplified DNA in the heated analysis sample; and
transmitting an analysis result of the DNA to the mobile device.
